(19) United States
(12) Patent Application Publication
(10) Pub. No.: US 2026/0076468 A1
(43) Pub. Date: Mar. 19, 2026

(54) CONTAINER HEAD WITH ELASTIC SEALING

(71) Applicant: SHENZHEN NUON MEDICAL EQUIPMENT CO., LTD., Shenzhen (CN)

(72) Inventors: Alain Dijkstra, Amstelveen (NL); LI XIANG, Shenzhen (CN); Tang Yuanzhou, Shenzhen (CN); Feng Jianxin, Shenzhen (CN); Mou Xiong, Shenzhen (CN); Wu Qingren, Shenzhen (CN)

(73) Assignee: SHENZHEN NUON MEDICAL EQUIPMENT CO., LTD., Shenzhen (CN)

(21) Appl. No.: 19/328,193

(22) Filed: Sep. 14, 2025

(30) Foreign Application Priority Data
Sep. 14, 2024 (CN) 2024112959978
Sep. 14, 2024 (CN) 2024222656820
Sep. 14, 2024 (CN) 2024222730496
Dec. 2, 2024 (CN) 2024229604901
Jun. 2, 2025 (CN) 2025211066330

(51) Int. Cl. A45D 34/04; A61H 23/02; A61N 5/06

(52) U.S. Cl. CPC A45D 34/041; A61H 23/02; A61N 5/0616; A45D 2200/205; A45D 2200/207; A61H 2201/0153; A61H 2201/105; A61N 2005/0644

(57) ABSTRACT

Embodiments of the invention relate to a container head designed for dispensing and applying liquid or semi-liquid skincare compositions. The container head includes a head body with a liquid inlet, a liquid outlet, and a channel connecting them. An elastic sealing structure, positioned at or near the outlet, comprises a sleeve body with a slit that normally remains closed to prevent leakage and elastically opens under pressure to allow controlled dispensing. A rolling ball applicator is rotatably mounted at the outlet in fluid communication with the sealing structure to deliver liquid to the skin during rolling contact. A detachable cover is provided to press the ball against the sealing structure, ensuring reliable sealing during storage and transport. Optional embodiments may include a flexible connection tube, a phototherapy lamp, and a vibration motor, thereby enabling multifunctional treatment and enhancing absorption of skincare formulations.

### TECHNICAL FIELD

The present invention relates to applicator technology for dispensing and applying liquid or semi-liquid skincare compositions. More particularly, the invention pertains to a container head incorporating leak-proof elastic sealing structures, a rolling ball applicator, and optional functional enhancements such as vibration and phototherapy, thereby enabling controlled, hygienic, and multifunctional delivery of cosmetic and dermatological products.

### BACKGROUND

Applicator heads for dispensing skincare products, cosmetics, and topical formulations are widely used. A common form of such devices is the ball-type applicator, in which a spherical element is mounted at the container outlet to spread liquid across the user's skin. While widely adopted, conventional applicator devices present significant shortcomings in terms of sealing performance, product control, and functional versatility.

A frequent drawback of traditional rolling ball applicators is leakage. Since the ball is only seated by friction within its housing, liquid may escape around its periphery during storage, transport, or pressure variations. This not only results in product loss but also raises hygiene concerns due to contamination.

Another limitation lies in dispensing control. Many known applicators fail to regulate the volume of product released, leading to excessive flow, uneven spreading, or dripping. The absence of integrated flow-blocking or anti-backflow structures further aggravates issues of dosing accuracy and container hygiene.

To address these concerns, improvements such as elastic sleeves or sealing caps have been proposed. However, these solutions are often implemented in isolation. A cap may improve sealing during storage, but does not control dispensing during use, while a flexible sleeve may reduce leakage but cannot guarantee reliable closure without supplementary mechanical support.

Moreover, consumer demand has evolved toward multifunctional skincare devices that not only deliver products but also provide enhanced treatment benefits. Modern treatment approaches integrate vibration massage, phototherapy, and other stimulation features to promote absorption and improve therapeutic outcomes. Conventional applicator heads are generally not suited for such integration, as they lack space for electrical components, transparent outlet structures for light emission, or stable mounting frameworks for vibration modules.

Therefore, there exists a need for a container head that overcomes the deficiencies of the prior art by combining leak-proof sealing mechanisms, controlled liquid dispensing, anti-backflow features, and mechanical closure systems with optional therapeutic enhancements such as vibration and phototherapy. Such a device should ensure secure storage, precise and hygienic delivery, and multifunctional skincare treatment within a single integrated structure.

### Objects of the Invention

An object of the present invention is to provide a container head for dispensing and applying liquid or semi-liquid skincare products with improved sealing performance, ensuring leak-proof storage and controlled dispensing.

Another object of the invention is to integrate a rolling ball applicator that not only spreads liquid evenly onto the skin but also provides a massage to enhance user comfort and promote absorption.

Another object of the invention is to incorporate an elastic sealing structure with a slit-based opening, which remains securely closed under non-use conditions and opens elastically under liquid pressure to prevent accidental leakage.

Yet another object of the invention is to provide a cover structure that mechanically presses the rolling ball against the sealing member to reinforce leak-proof storage and safe transport.

A still further object of the invention is to introduce a flexible tube connection between the liquid inlet end and the liquid channel, thereby simplifying assembly, reducing manufacturing complexity, and improving sealing reliability.

Another object of the invention is to provide optional stimulation elements, including a vibration motor for mechanical massage and a phototherapy lamp for light-based treatment, to enhance skincare efficacy.

Another object of the invention is to house the stimulation elements, battery, and control circuitry within a compact structural arrangement of outer shell, inner shell, support frame, and bottom cover, ensuring efficient use of space and convenient integration.

It is also an object of the invention to provide an applicator system that combines product application, leak-proof storage, and multifunctional skincare treatment within a single, ergonomically designed device.

### SUMMARY OF THE INVENTION

According to a first aspect of the present invention, a container head for dispensing liquid or semi-liquid compositions is provided. The container head comprising: a head body having a liquid inlet end configured to be coupled to a liquid storage container and a liquid outlet end; a liquid channel extending between the liquid inlet end and the liquid outlet end; an elastic sealing structure mounted at or adjacent to the liquid outlet end, the elastic sealing structure is provided with a slit, the slit being normally closed to prevent leakage of liquid from the liquid storage container and elastically deformable to open under liquid pressure to permit flow through the liquid outlet end; and a rolling ball applicator rotatably received in a groove at the liquid outlet end, the rolling ball being positioned to receive liquid discharged through the slit and to apply the liquid to a user's skin during rolling contact.

In one embodiment of the invention, the slit is selected from a linear cut, a cross cut, a T-shaped cut, or a V-shaped cut.

In one embodiment of the invention, the elastic sealing structure comprises a flexible elastomeric material.

In one embodiment of the invention, the elastic sealing structure comprises a blocking portion integrally formed with a mounting portion, the blocking portion enclosing the slit that opens under liquid pressure and closes when the pressure is removed.

In one embodiment of the invention, the elastic sealing structure comprises a sleeve having an end wall with the slit that elastically deforms under pressure to permit liquid flow and returns to a closed state when the pressure is removed.

In one embodiment of the invention, the slit is provided on the sleeve of the elastic sealing structure.

In one embodiment of the invention, the liquid channel further comprises a second elastic sealing structure positioned upstream of the elastic sealing structure to prevent backflow toward the liquid storage container.

In one embodiment of the invention, the ball applicator is mounted in a spherical groove formed in a ball seat of the main body and configured to roll relative to the spherical groove.

According to a second aspect of the invention, a multifunctional applicator system is provided. The multifunctional applicator system comprising: a container head having a liquid inlet end, a liquid outlet end, and a liquid channel extending therebetween; an elastic sealing structure disposed at or adjacent to the liquid outlet end, the elastic sealing structure being normally closed to prevent liquid leakage and elastically deformable to permit liquid flow under applied pressure; a rolling ball applicator rotatably mounted at the liquid outlet end in fluid communication with the elastic sealing structure for dispensing liquid onto a user's skin; and a detachable cover configured to press the rolling ball against the elastic sealing structure when fitted, thereby reinforcing sealing during storage and transport.

In one embodiment of the invention, the cover includes an inner protrusion configured to press the rolling ball into the elastic sealing structure.

In one embodiment of the invention, the multifunctional applicator system further comprises an outer shell and an inner shell assembled to define a cavity for accommodating internal components.

In one embodiment of the invention, the multifunctional applicator system further comprising at least one stimulation element selected from a phototherapy lamp and a vibration motor.

In one embodiment of the invention, the elastic sealing structure comprises an elastic flap oriented toward the ball applicator to promote rapid closure after liquid pressure is released.

In one embodiment of the invention, the elastic sealing structure comprises a flexible sleeve having an end wall with a slit selected from a straight line slit, a cross-shaped slit, or a T-shaped slit.

In one embodiment of the invention, the slit is provided on the sleeve of the elastic sealing structure.

In one embodiment of the invention, the multifunctional applicator system further comprising a second elastic sealing structure disposed upstream of the liquid outlet to provide dual leakage prevention.

According to a third aspect of the invention, a method of dispensing and applying a liquid skincare composition using a container head is provided. The method comprising: coupling the container head to a liquid storage container at a liquid inlet end; applying pressure to the liquid storage container such that liquid flows into a liquid channel of the container head; elastically deforming an end wall of a elastic sealing structure under liquid pressure to open a slit and discharge the liquid toward a rolling ball applicator; rolling the rolling ball applicator against a user's skin to transfer and spread the liquid thereon.

In one embodiment of the invention, applying pressure to the liquid storage container comprises squeezing a flexible bottle.

In one embodiment of the invention, the rolling ball applicator simultaneously massages the skin while transferring the liquid.

In one embodiment of the invention, the method further comprising covering the rolling ball applicator with the detachable cover after use to mechanically reseal the slit of the elastic sealing structure.

In the context of the specification, the term “comprising” (and grammatical variations thereof) is intended to be open-ended, meaning that the listed elements or steps are not exhaustive and do not exclude the presence of additional, unrecited elements or steps.

In the context of the specification, the terms “a,” “an,” and “the” are intended to cover both the singular and plural forms, unless otherwise indicated by context.

In the context of the specification, the term “or” is used inclusively, unless stated otherwise, meaning “and/or.”

In the context of the specification, the terms such as “axial,” “circumferential,” “radial,” “inner,” “outer,” “upper,” “lower,” “front,” “rear,” and the like are used for convenience to describe relative orientation and do not impose any absolute limitations on device orientation during use.

In the context of the specification, the term “container head” broadly refers to a dispensing assembly, applicator head, cap structure, or any equivalent component configured to be mounted to a liquid storage container for controlled dispensing of liquid or semi-liquid compositions.

In the context of the specification, the term “liquid inlet end” refers to a portion of the container head configured to connect to, couple with, or otherwise communicate with a liquid storage reservoir. The term “liquid outlet end” refers to a portion of the container head from which liquid exits toward an applicator, sealing member, or user's skin.

In the context of the specification, the term “liquid channel” refers to any passage, conduit, or pathway that enables fluid communication between the liquid inlet end and the liquid outlet end. The channel may be formed integrally in a head body, defined by multiple assembled parts, or include flexible conduits such as tubes.

In the context of the specification, the term “elastic sealing structure” broadly encompasses any flexible, resilient, or deformable member configured to normally block the liquid channel or outlet, and to elastically deform or displace under liquid pressure to permit controlled flow. Examples include sleeves, diaphragms, flaps, check valves, membranes, or equivalent elements with one or more slits, openings, or deformable portions.

In the context of the specification, the term “slit” (also referred to as opening structure) includes any cut, notch, perforation, or thinned portion in the elastic sealing structure that remains closed in the absence of pressure and opens elastically under applied liquid pressure. The slit may be linear, cross-shaped, V-shaped, T-shaped, arcuate, or of other configurations.

In the context of the specification, the term “rolling ball applicator” (also referred to as first ball or second ball) refers to a spherical applicator element rotatably received in a groove, housing, or retaining seat, and configured to transfer liquid to a user's skin during rolling contact.

In the context of the specification, the term “cover” (or top cap) refers to any detachable or movable member configured to cover the liquid outlet end of the container head. The cover may be threaded, snapped, hinged, or otherwise removably attached, and may include inner protrusions or retaining structures that press the rolling ball against the elastic sealing structure to mechanically seal the outlet during non-use.

In the context of the specification, the term “support structure” refers to any column, post, frame, or rib positioned within or adjacent to the liquid channel or sealing element to provide support, limit displacement, or reinforce closure of the slit under compressive force.

In the context of the specification, the term “stimulation element” includes but is not limited to phototherapy lamps, vibration motors, heating modules, cooling modules, or other components that provide therapeutic, cosmetic, or sensory effects in combination with liquid application.

In the context of the specification, the term “flexible tube” or “connection assembly” refers to an elastic conduit, hose, or tube member configured to establish fluid communication between two portions of the liquid channel or between the container head and a liquid reservoir, providing sealed, flexible, and shock-absorbing connectivity.

In the context of the specification, the term “user's skin” includes facial skin, scalp, neck, or other external body surfaces where topical application of the liquid or semi-liquid composition is intended.

### BRIEF DESCRIPTION OF THE ACCOMPANYING DRAWINGS

In order to more clearly illustrate the technical solutions in the embodiments of the present invention, the drawings that need to be used in the embodiments or prior art descriptions will be briefly introduced below, and it is obvious that the drawings in the following descriptions are only some embodiments of the present invention, and for those skilled in the art, other drawings can also be obtained according to these drawings without paying creative labor.

FIG. 1 is a schematic cross-sectional view of a container head, in accordance with an embodiment of the present invention.

FIG. 2 is an enlarged schematic view of a liquid channel of the container head, in accordance with an embodiment of the present invention.

FIG. 3A is a schematic three-dimensional view of an elastic sealing structure, in accordance with an embodiment of the present invention.

FIG. 3B is a bottom perspective three-dimensional view of the elastic sealing structure, in accordance with an embodiment of the present invention.

FIG. 4 is a schematic cross-sectional view of a second elastic sealing structure, in accordance with an embodiment of the present invention.

FIG. 5 is a schematic cross-sectional view of a ball seat provided in the present application.

FIG. 6 is a schematic diagram showing the elastic sealing structure in a second type of container head, in accordance with an embodiment of the present invention.

FIG. 7 is a schematic diagram of the structure of the elastic sealing structure, in accordance with another embodiment of the present invention.

FIG. 8 is a schematic cross-sectional view of the elastic sealing structure of FIG. 8, in accordance with another embodiment of the present invention.

FIG. 9 is a schematic diagram showing the elastic sealing structure in another container head, in accordance with an embodiment of the present invention.

FIG. 10 is a schematic structural diagram of the elastic sealing structure, in accordance with an embodiment of the present invention.

FIG. 11 is a schematic structural diagram of the elastic sealing structure with a slit, in accordance with an embodiment of the present invention.

### DETAILED DESCRIPTION

Embodiments of the present invention disclosure will be described more fully hereinafter with reference to the accompanying drawings in which like numerals represent like elements throughout the figures, and in which example embodiments are shown.

The detailed description and the accompanying drawings illustrate the specific exemplary embodiments by which the disclosure is practiced. These embodiments are described in detail to enable those skilled in the art to practice the invention illustrated in the disclosure. It is to be understood that other embodiments are utilized, and other changes are made, without departing from the spirit or scope of the present disclosure. The following detailed description is therefore not to be taken in a limiting sense, and the scope of the present invention disclosure is defined by the appended claims. Embodiments of the claims, however, may be embodied in many different forms and should not be construed as limited to the embodiments set forth herein.

It should be noted that when an element is referred to as being “fixed to” or “disposed on” another element, it is directly or indirectly attached to the other element. When an element is referred to as being “connected to” another element, it is directly or indirectly connected to the other element.

Embodiment of the present invention discloses a container head for dispensing and applying liquid or semi-liquid skincare compositions. The container head integrates leak-proof sealing structures, rolling ball applicators, flexible tube connections, and optional stimulation features such as vibration and phototherapy. The container head is adapted to be mounted on a liquid storage container and is configured to ensure secure storage without leakage while permitting controlled dispensing and enhanced treatment during application.

In one embodiment, the container head comprises a head body provided with a liquid inlet end configured for attachment to a liquid storage container, and a liquid outlet end configured to deliver liquid to the skin. A liquid channel extends between the inlet end and the outlet end. A first elastic sealing structure is arranged adjacent to the liquid outlet. The sealing structure includes a mounting portion fixed to the head body and a blocking portion formed as an elastic flap. The flap normally closes a first discharge port, and under liquid pressure deforms to open the port and allow liquid to pass toward the outlet.

A first ball is mounted at the liquid outlet end within a spherical groove formed in the head body. The first ball faces the elastic flap such that, in its resting state, the ball presses against the blocking portion to maintain the discharge port closed. The first ball is partially exposed externally to contact and roll against the user's skin, thereby transferring the dispensed liquid evenly.

To further improve sealing performance, a second elastic sealing structure may be disposed between an inlet section and an outlet section of the liquid channel. This second sealing structure comprises a cylindrical portion carrying a second blocking portion provided with a second discharge port. In one implementation, the second blocking portion is a flexible flap or check valve permitting downstream liquid flow while preventing backflow toward the liquid reservoir, thereby ensuring product hygiene.

In a preferred configuration, a detachable cover is provided at the liquid outlet end of the head body. The cover may be threaded or snap-fitted to the head body and is furnished with an inner abutment protrusion. When the cover is mounted, the protrusion presses the first ball tightly against the elastic sealing structure, thereby mechanically closing the discharge port and preventing leakage during storage or transport.

In another embodiment, the container head comprises a main body having a liquid inlet end, a liquid outlet, and a liquid channel extending therebetween. A sealing member in the form of a flexible sleeve is mounted at the outlet. The sleeve has an open end secured to the outlet and a closed end provided with an opening structure. The opening structure may take the form of a straight slit, strip, cross-shaped, T-shaped, or V-shaped cut, which remains closed under normal conditions and elastically opens only when liquid pressure is applied.

A housing is arranged at the outlet to accommodate the first ball. The housing defines a groove in which the ball is rotatably received and an aperture through which the ball partially protrudes. The ball abuts the closed end of the flexible sleeve such that, when the slit opens under liquid pressure, the liquid flows around the ball and is spread onto the skin during rolling motion.

A cover portion may be detachably secured over the housing. The cover portion is provided with a limiting column that, when installed, presses the ball firmly against the sealing member. In this state, the opening structure is mechanically restricted from opening, thereby ensuring reliable sealing during non-use. To reinforce this action, one or more support columns may be disposed in the liquid channel opposite the sealing member. When the cover presses the ball into the sealing member, the slit is further supported by the column and maintained in a tightly closed state.

In another embodiment, the container head comprises an outer shell, an inner shell, and a bottom cover assembled together to form a cavity for accommodating functional components. A liquid channel is formed within the inner shell and connected to a liquid inlet formed on the bottom cover via a flexible tube. The flexible tube has sealing ends mounted on flanges provided respectively on the inner shell and the bottom cover. This tube connection provides an elastic and reliable sealing arrangement that minimizes leakage risk while simplifying molding of the inner shell.

Adjacent to the outlet of the inner shell, a sealing sleeve is installed. The sleeve includes a sleeve body, an end wall, and a slit formed in the end wall. The slit remains normally closed and opens elastically under liquid pressure to permit controlled discharge. A first ball is mounted in a rolling groove of an end cap secured at the outlet, the end cap being at least partially light-transmissive. A detachable top cover is provided over the end cap and is furnished with an inner abutment protrusion that presses the ball against the end wall of the sealing sleeve when the cover is closed, thereby preventing inadvertent opening of the slit.

A support frame may be mounted within the outer shell. The support frame assists in positioning the flexible tube and also serves as a mounting structure for stimulation elements. These may include, for example, a phototherapy lamp arranged to emit light through the translucent end cap toward the skin, or a vibration motor configured to impart oscillatory motion to the container head. The phototherapy lamp may be selected to emit violet, blue, red, infrared, or other therapeutic wavelengths. A battery and control circuit board are disposed within the cavity defined by the outer shell and bottom cover to power and regulate the stimulation elements.

In operation, when the container head is in a storage state, the cover presses the ball into the sealing member so that the slit or discharge port remains closed. Upon removal of the cover and application of pressure to the liquid container, the sealing member elastically deforms to open, thereby permitting liquid to flow through the channel and onto the first ball. The ball then applies the liquid uniformly to the user's skin while rolling. If activated, the phototherapy lamp irradiates the skin and the vibration motor imparts mechanical massage, thereby enhancing product absorption and therapeutic effect. When pressure is released, the sealing member promptly returns to its closed state, and re-installation of the cover ensures additional mechanical sealing.

Several embodiments of the present invention will now be described in detail with references to FIGS.

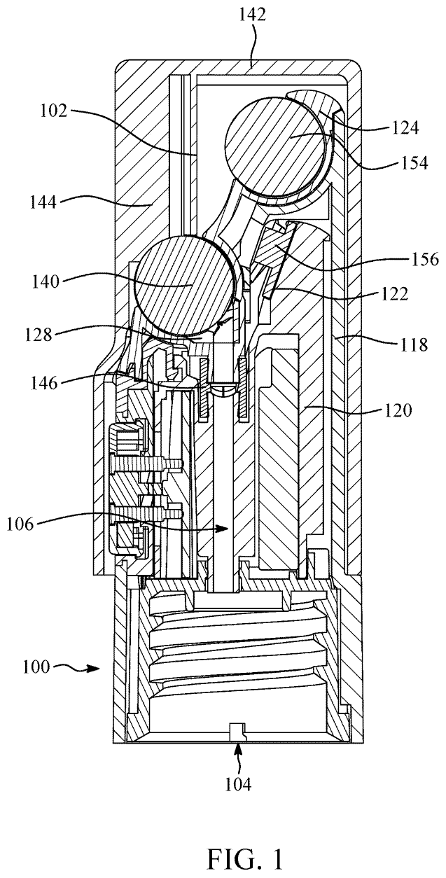

Referring now to FIG. 1, the present invention discloses a container head structure comprising a container head 100. The container head 100 is provided with a liquid inlet end 104 and a liquid outlet end 102. The liquid inlet end 104 is configured to connect with a liquid storage element (not shown), such as a cosmetic reservoir, essential oil container, or medicinal solution bottle. A liquid channel 106 extends between the liquid inlet end 104 and the liquid outlet end 102 so as to enable communication between the liquid storage element and the dispensing region of the liquid supply head. The application liquid stored in the reservoir flows from the liquid inlet end 104, through the liquid channel 106, and ultimately exits via the liquid outlet end 102.

To address the problem of liquid leakage, an elastic sealing structure is disposed along the liquid channel 106. The elastic sealing structure selectively blocks the flow of the coating liquid and is provided with a slit that may be switched between an open state and a closed state, thereby enabling or disabling fluid communication between the liquid channel 106 and the external environment. In some embodiments, the elastic sealing structure is a resilient, elastic sealing member that deforms in response to fluid pressure. The elastic sealing structure does not permanently obstruct the liquid channel; rather, it maintains a closed position under normal conditions but opens elastically under the internal pressure generated when the reservoir is squeezed. Once pressure subsides, the elastic sealing structure returns to its original closed configuration, thereby preventing unintended leakage.

In alternative embodiments, the elastic sealing structure may be implemented as an electronic valve that opens and closes the slit in response to electrical control signals. In yet another embodiment, the elastic sealing structure may be a manually operated valve that can be selectively actuated by the user. In contrast to conventional container heads, which lack an elastic sealing structure and are prone to leakage through the clearance between the dispensing ball and the housing, the present invention introduces an elastic sealing structure within the liquid channel to ensure reliable sealing and leakage prevention.

According to one embodiment, the elastic sealing structure is flexible and elastic, and is specifically provided with a slit-type drainage port. When the reservoir is squeezed, the pressure of the coating liquid forces the slit to open elastically, permitting the liquid to flow through. Once the squeezing force is released, the slit closes immediately due to the elastic restoring force, thus preventing further liquid discharge.

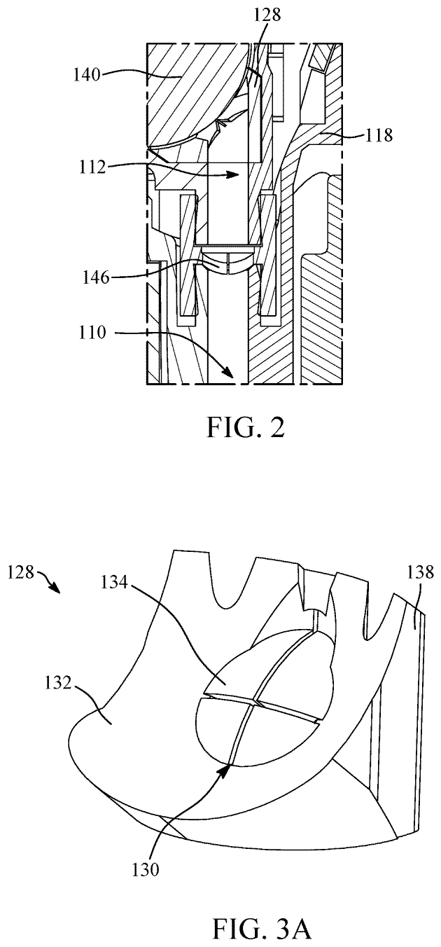

As shown in FIGS. 1 and 2, the liquid channel 106 comprises a liquid inlet section 110 and a liquid outlet section 112. At the liquid outlet 108, an elastic sealing structure 128 is disposed. This elastic sealing structure defines a slit 130 and cooperates with a first ball 140 rollably mounted at the liquid outlet end 102. The first ball 140 faces the elastic sealing structure 128 such that, when pressed against the elastic sealing structure, it blocks the slit 130 and seals the channel, thereby further enhancing leakage prevention.

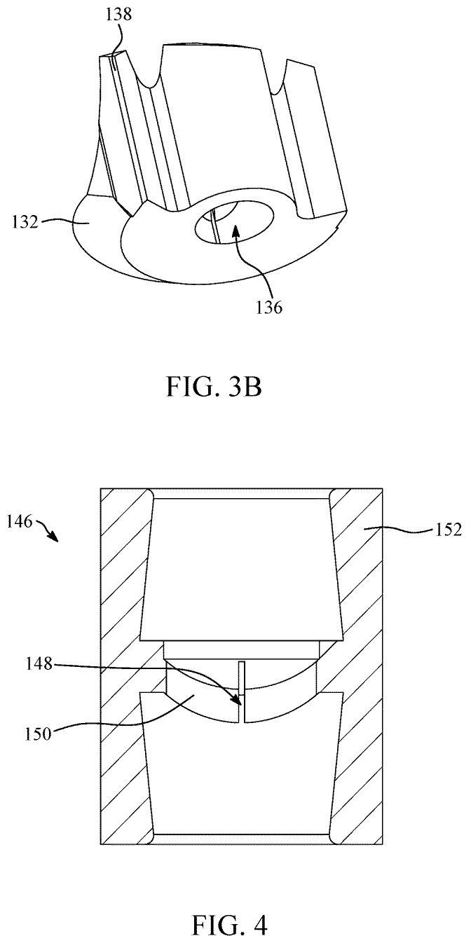

Referring to FIGS. 1 and 3, the elastic sealing structure 128 includes a mounting portion 132 and a first blocking portion 134. The mounting portion 132 is fixed to the container head 100 and defines a connecting channel 136, which communicates with the liquid channel 106. The first blocking portion 134, which covers the connecting channel 136, is a flexible flap integrally formed with the mounting portion 132. The edges of the flap enclose the slit 130. When subjected to pressure, the flap deforms to open the slit 130 and allows fluid to pass. Once pressure subsides, the flap returns to its closed position. When the first ball 140 presses against the elastic sealing structure, it contacts both the mounting portion 132 and part of the flap, ensuring that the slit 130 remains sealed regardless of the flap's open or closed state. To improve sealing and elastic recovery, the first blocking portion 134 is shaped with a curved surface protruding toward the first ball 140.

As shown in FIG. 1, and 5, a first spherical groove 114 is provided on the container head 100 to accommodate rolling of the first ball 140. A mounting groove 116 is arranged at the base of the spherical groove 114, and the elastic sealing structure 128 is embedded therein. In certain embodiments, the elastic sealing structure is further equipped with a retaining rib 138, while the mounting groove 116 is correspondingly provided with a retaining groove. This improves stability, enlarges the contact area, and prevents accidental displacement of the barrier.

Referring to FIG. 1, the container head further includes a cover 142 detachably secured to the container head 100 by means such as threading or snap-fit. The cover 142 is provided with an abutment portion 144 that presses against the first ball 140 when the cover is mounted. This action forces the ball into tight contact with the elastic sealing structure 128, thereby closing the slit 130 and preventing liquid leakage during storage or transport.

As shown in FIG. 2, a second elastic sealing structure 146 is disposed between the liquid inlet section 110 and the liquid outlet section 112 of the liquid channel 106. The second elastic sealing structure 146 includes a second blocking portion 150 defining a second slit 148. The second elastic sealing structure provides an additional level of sealing, ensuring that liquid cannot backflow into the reservoir and thereby preventing contamination of the stored liquid. The second elastic sealing structure 146 may also be a flexible flap integrally formed with a cylindrical portion 152. The flap encloses the second slit 148, opening elastically under fluid pressure and closing once the pressure subsides. In preferred embodiments, the flap is concave toward the liquid inlet section 110, ensuring rapid return to the closed state.

As shown in FIG. 4, the cylindrical portion 152 connects the liquid outlet section 112 and the liquid inlet section 110, with the second blocking portion 150 arranged between them. Insertion of the cylindrical portion 152 into both the housing 118 and the mounting bracket 120 provides stable integration of the second elastic sealing structure 146.

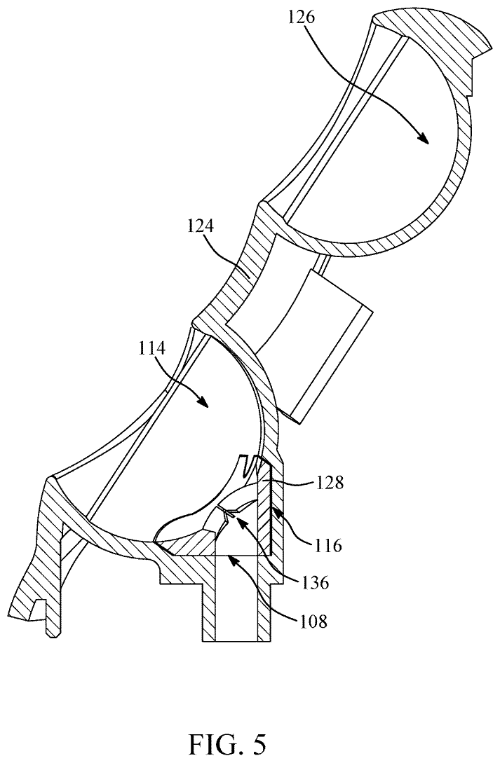

Referring to FIG. 5, the container head further comprises a ball seat 124 fixed to the housing 118. The ball seat 124 defines the first spherical groove 114 for the first ball 140 and may also include a second spherical groove 126, within which a second ball 154 is rotatably mounted. Unlike the first ball 140, the second ball 154 does not dispense liquid but serves only for skin rolling. To allow the cover 142 to simultaneously enclose both balls, the first ball 140 and the second ball 154 are arranged in staggered axial and radial positions. This arrangement not only shortens the liquid channel 106, reducing liquid waste, but also enhances dispensing efficiency.

In an embodiment, the mounting bracket 120 also supports one or more stimulation components, such as a vibrator, phototherapy lamp 156, or heating element. These elements promote microcirculation, accelerate absorption of the dispensed liquid, and enhance skincare efficacy. For example, when the phototherapy lamp 156 is mounted on the mounting bracket 120, it is positioned at a mounting bevel 122 arranged at an angle corresponding to the diagonal alignment of the first and second balls. The ball seat 124 is formed of a translucent material to enable light transmission, and the phototherapy lamp 156 is positioned between the two balls to ensure that the emitted light is not obstructed.

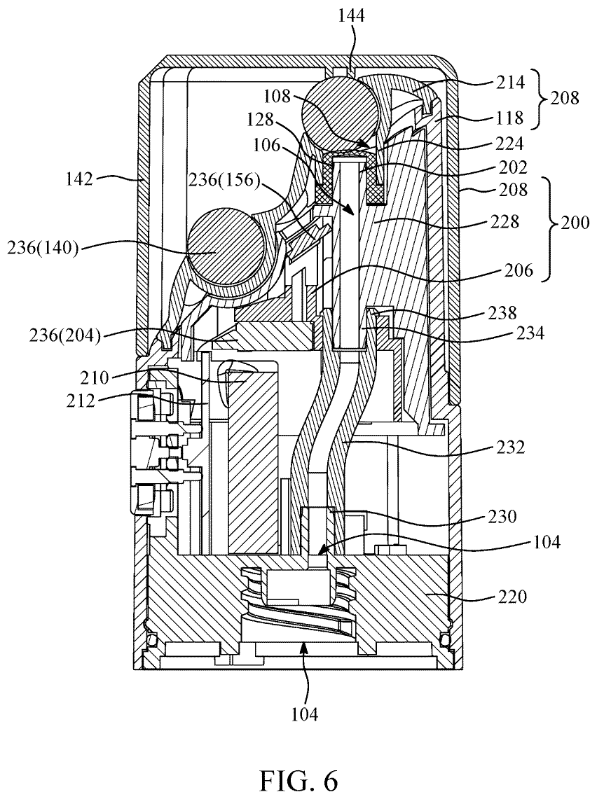

Referring to FIG. 6, the elastic sealing structure to control the leakage of the liquid is shown on an alternate configuration of the container head configured to be mounted on a liquid container. When assembled, the skincare product stored in the liquid container flows through the container head 100, and a stimulation element 236 mounted thereon stimulates the skin, thereby promoting enhanced absorption of the product.

The container head 100 comprises a main body 200, a bottom cover 220, a flexible tube 232, and the stimulation element 236. The main body 200 is provided with the liquid outlet 108 on its outer surface. The liquid channel 106 is defined inside the main body 200, one end of which is sealed to the liquid outlet 108, while the opposite end remains open. The bottom cover 220 encloses the open end of the main body 200 and includes the liquid inlet end 104. The liquid inlet end 104 enables the bottom cover 220 to be secured to the liquid container. The flexible tube 232 connects the liquid channel 106 and the liquid inlet end 104, its two ends being sealed to each other. The stimulation element 236 is mounted on the main body 200.

In this embodiment, the connection between the liquid channel 106 and the liquid inlet end 104 via the flexible tube 232 ensures that the elasticity of the flexible tube 232 maintains a tight fit at both ends. This significantly reduces the risk of leakage at the junctions with the main body 200 and bottom cover 220. During use, when the skincare product flows through the liquid channel 106, exits via the liquid outlet 108, and is applied to the skin, the stimulation element 236 provides mechanical or energy-based stimulation to promote absorption of the product.

In some embodiments, the main body 200 comprises an outer shell 208 and an inner shell 228. The outer shell 208 is a hollow member provided with the liquid outlet 108, and its distal end is enclosed by the bottom cover 220. The inner shell 228, housed within the outer shell 208, defines the liquid channel 106. In alternative embodiments, the liquid channel 106 may be directly formed in the outer shell 208. The outer shell 208 and inner shell 228 cooperate structurally to reinforce each other.

The inner shell 228 is a hollow structure having one closed end and one open end. The closed end faces toward the liquid outlet 108, while the open end faces the bottom cover 220 and is supported thereon. To prevent relative rotation between the inner shell 228 and the bottom cover 220, the bottom cover 220 may be provided with a latch, while the inner shell 228 includes a corresponding slot that engages with the latch.

A space is defined between the inner shell 228 and the bottom cover 220, within which various functional components may be housed. For example, in some embodiments, the container head 100 further comprises a battery 210 and a control board 212, both disposed in the space and fixed to the bottom cover 220. After securing these components, the inner shell 228 is assembled onto the bottom cover 220 to enclose and protect them. To aid in fixation, the bottom cover 220 may include ribs with multiple slots for accommodating the ends of the battery 210 and control board 212.

Compared to directly extending the liquid channel 106 from the inner shell 228 to the liquid inlet end 104, the use of the flexible tube 232 provides several advantages: (i) it avoids the need to lengthen the inner shell 228, thereby reducing material usage and simplifying manufacturing; (ii) it leaves more internal volume available for installation of additional structures; and (iii) it enhances spatial efficiency within the container head 100.

In certain embodiments, the inner shell 228 includes a first flange 234 of annular form, around which a portion of the liquid channel 106 is defined. One end of the flexible tube 232 is sealingly mounted on the first flange 234. The bottom cover 220 may include a second flange 230 surrounding the liquid inlet end 104, onto which the opposite end of the flexible tube 232 is sealingly mounted.

In other embodiments, one end of the flexible tube 232 is provided with a barb 238 that engages the main body 200 to prevent separation. Specifically, when a support frame 206 is provided, it is fixed to the inner shell 228 and includes a through-hole through which the first flange 234 and the flexible tube 232 pass. The flexible tube 232 is clamped between the through-hole wall and the first flange 234, while the barb 238 hooks onto the support frame 206, preventing disengagement. The support frame 206 and inner shell 228 are separately manufactured, thereby simplifying fabrication. Moreover, the support frame 206 provides radial clamping and axial positioning for the flexible tube 232.

In some embodiments, the inner shell 228 is relatively large to provide structural support for the outer shell 208. The support frame 206 is positioned within the inner shell 228 in a compact manner to maximize space utilization. Preferably, the support frame 206 is located away from the bottom cover 220, ensuring sufficient space for accommodating the battery 210 and related electronics.

The stimulation element 236 may comprise a phototherapy lamp 156. In such embodiments, the inner shell 228 acts as a light shield, while its closed end is provided with a light-transmitting aperture. The phototherapy lamp 156, mounted on the support frame 206, is aligned with the aperture so that emitted light passes outward for skin phototherapy. The lamp may emit one or more wavelengths, including but not limited to violet, blue, red, or infrared light. The support frame 206 thus provides a convenient mounting structure for the lamp, reducing the design complexity of the inner shell 228 while also concealing internal components from external view.

The support frame 206 may include support ribs with inclined support steps. A stop rib is arranged below the support steps. The phototherapy lamp 156 rests on the support steps and is restrained in position by the stop rib.

In some embodiments, the housing comprises a housing 118 and a light-transmissive end cap 214. The bottom cover 220 is mounted to one end of the housing 118, while the end cap 214 is fixed to the opposite end. Light emitted from the lamp passes through the end cap 214, while the housing 118 acts as a light shield to conceal the interior. To ensure stable assembly, the end cap 214 may engage an annular groove formed in the housing 118, optionally bonded with adhesive for a sealed connection.

In addition, the stimulation element 236 may further comprise a first ball 140. The end cap 214 defines a rolling groove within which the first ball 140 is partially accommodated in rolling contact. The liquid outlet 108 opens into the rolling groove, enabling the skincare product to flow into the groove and adhere to the surface of the first ball 140. During rolling motion, the first ball 140 both dispenses product and massages the skin. Simultaneously, the phototherapy lamp 156 provides phototherapy through the end cap 214.

The stimulation element 236 may also include a vibration motor 204 fixed to the support frame 206. The vibration motor 204 imparts vibration to the container head 100, thereby enhancing massage and improving product absorption.

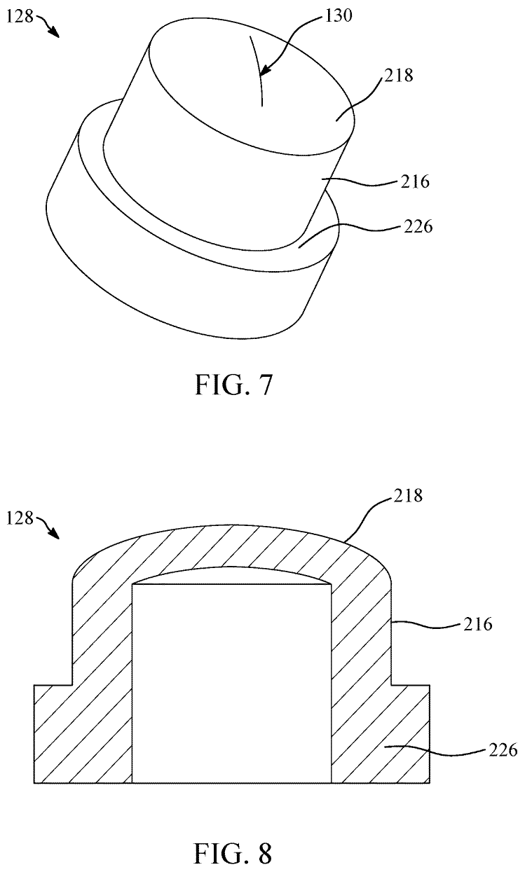

Referring to FIGS. 6 to 8, the container head 100 further includes an elastic sealing structure 128. The inner surface of the outer shell 208 is provided with a third flange 224 surrounding the liquid outlet 108. The inner shell 228 includes a fourth flange 202 forming part of the liquid channel 106. The elastic sealing structure 128 comprises a sleeve 216, an end wall 218, and a retaining stopper 226. The end wall 218 is provided with a slit 130, while the stopper 226 projects from the circumference of the sleeve 216. The elastic sealing structure 128 is mounted with the sleeve 216 on the fourth flange 202, while the third flange 224 abuts the stopper 226, thereby securing the elastic sealing structure 128 in position.

In an embodiment, the main body 200 is provided with a bracket 120, a first connecting plate 312, and a second connecting plate 314, the first connecting plate 312 and the second connecting plate 314 being arranged in a stacked configuration. The main body 200 further comprises a liquid channel 106, the liquid channel 106 including a first branch structure 302 formed within the bracket 120 and a second branch structure 304 formed within the first connecting plate 312 and the second connecting plate 314. The bracket 120 defines a liquid inlet end 104 that is in fluid communication with the first branch structure 302. The first connecting plate 312 defines a liquid outlet 108 that is in fluid communication with the second branch structure 304. The second connecting plate 314 further defines a through-hole that establishes fluid communication between the first branch structure 302 and the second branch structure 304.

When the liquid container is squeezed, the internal pressure deforms the end wall 218, causing the slit 130 to elastically open and allow product flow toward the liquid outlet 108. Upon release of pressure, the end wall 218 restores its shape, and the slit 130 closes, preventing leakage. The slit 130 is formed by cutting into the end wall 218 without removing material, ensuring that in its natural state, it remains closed. The slit 130 may be straight, cross-shaped, or T-shaped. Optionally, the end wall 218 may be outwardly arched to facilitate contact with the first ball 140.

As shown in FIG. 6, the container head 100 may also include a cover 142 enclosing the first ball 140. An abutment portion 144 formed on the inner wall of the cover 142 presses the first ball 140 against the end wall 218, thereby preventing the slit 130 from opening under static pressure and avoiding leakage during storage or transport.

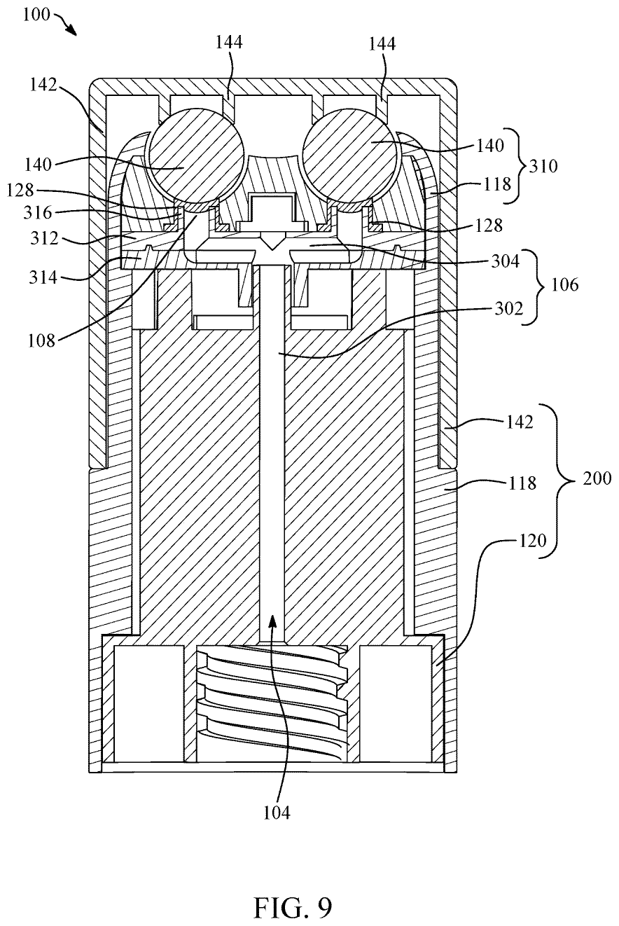

Referring to FIG. 9, the elastic sealing structure to control the leakage of the liquid is shown on an alternate configuration of the container head. The container head 100 comprises a main body 200, an elastic sealing structure 128, a contact portion 310, and a cover 142. The main body 200 serves as the primary structural component of the container head 100 and provides support for other components. The cover 142 functions to enclose the main body 200 when the container head 100 is not in use, thereby preventing leakage and contamination. The contact portion 310 is disposed adjacent to the liquid outlet 108 of the main body 200 and is adapted to deliver the skincare product uniformly. The elastic sealing structure 128 is positioned between the liquid outlet 108 and the contact portion 310, ensuring that product leakage is minimized when the container head is in a storage state.

The main body 200 includes a liquid inlet end 104, a liquid outlet 108, and a liquid channel 106 extending therebetween. The liquid inlet end 104 communicates with a liquid reservoir (not shown), such as a cream or lotion container, and directs the skincare product into the liquid channel 106. The liquid channel 106 guides the flow of the skincare product toward the liquid outlet 108, where it is ultimately dispensed to the user's skin.

Referring to FIG. 9, and 10, the elastic sealing structure 128 is of flexible construction and has an open end 308, an end wall 218, and a sleeve 216 extending therebetween. The open end 308 is mounted over the liquid outlet 108 of the main body 200, while the end wall 218 corresponds to the contact portion 310. A slit 130 is provided in the sleeve 216 of the elastic sealing structure 128. In one variation, the slit 130 is a strip-shaped slit extending circumferentially around the sleeve 216. This arrangement allows the skincare product to flow outward when the liquid reservoir is squeezed, while maintaining sealing performance in the absence of pressure.

To provide support for the flexible elastic sealing structure 128, the main body 200 is equipped with a support post 316. The support post 316 projects outwardly from the main body and provides a mounting surface for the elastic sealing structure 128. In operation, when the liquid reservoir is compressed, the skincare product exerts pressure against the inner surface of the elastic sealing structure 128, causing the slit 130 to elastically deform and open. The product then flows through the liquid channel 106 and exits through the contact portion 310 for application.

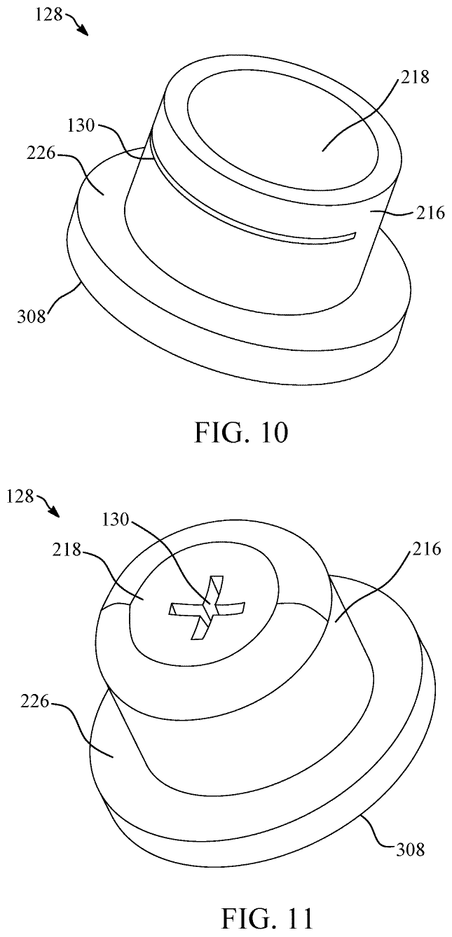

Referring to FIGS. 9 to 11, the slit 130 is disposed at the end wall 218 of the elastic sealing structure 128 rather than on the sleeve 216. In this configuration, the slit 130 may be cross-shaped, although other shapes, such as T-shaped, V-shaped, or straight slits, may also be employed.

A support column can be provided on the second connecting plate 314, which can be positioned within the liquid channel 106 of the main body 200. One end of the support column extends outward from the liquid outlet 108 into contact with the end wall 218 of the elastic sealing structure 128. When the elastic sealing structure 128 is subjected to compressive forces from the skincare product, the end wall 218 abuts the support column, causing the cross-shaped slit 130 to close. When the compressive force is released, the end wall 218 separates from the support column, allowing the slit 130 to reopen. This arrangement provides a self-regulating sealing mechanism that prevents unintended leakage when the device is not in use.

In an embodiment, the contact portion 310 comprises a housing 118 and a first ball 140. The housing 118 is mounted to the main body 200 and retains the first ball 140 in a movable manner, allowing the ball to rotate during application. The first ball 140, directly contacts the skin of the user and ensures even distribution of the skincare product. The first ball 140 is positioned against the elastic sealing structure 128 so that, when pressure is applied to the reservoir, product passes through the slit 130 and is dispensed around the ball for smooth application.

In some embodiments, the cover 142 includes an abutment portion 144 on its inner wall. When the cover 142 is fitted onto the main body 200, the abutment portion 144 presses the first ball 140 against the elastic sealing structure 128. This force prevents the elastic sealing structure 128 from elastically deforming, thereby keeping the slit 130 closed and avoiding leakage.

The cover 142 is configured with both an open state and a closed state. In the closed state, the cover 142 encloses the main body 200, applies a retaining force on the first ball 140 via the abutment portion 144, and prevents deformation of the elastic sealing structure 128. This effectively seals the liquid outlet 108 and ensures that no skincare product leaks during storage or transportation.

In the open state, the cover 142 is removed from the main body 200, thereby releasing the compressive force on the first ball 140 and allowing the elastic sealing structure 128 to elastically deform. In this condition, when the reservoir is compressed, pressure in the liquid channel 106 acts on the elastic sealing structure 128, opening the slit 130, and permitting product to flow toward the first ball 140 for application to the skin.

It should be understood that the slit 130 may be positioned on the sleeve 216, on the end wall 218, or on both. The slit 130 may take the form of a straight slit, a cross-shaped slit, a T-shaped slit, or a V-shaped slit, and may be sized or oriented to adjust the flow characteristics. The elastic sealing structure 128 may be disc-shaped, plate-shaped, cover-shaped, or irregular in form, depending on the design of the container head.

The contact portion 310 may incorporate one or more first balls 140, or may be formed with alternative applicator elements such as rollers, vibrating elements, or textured pads. The cover 142 may be hinged, threaded, or snap-fit to the main body 200.

In an embodiment, the invention further provides a liquid supply device comprising a liquid reservoir and the above-described container head structure. The reservoir may be a bottle, flexible hose, or equivalent storage container, connected to the head by threaded, snap-fit, or plug-in means. In one embodiment, the reservoir includes external threads while the liquid inlet end 104 of the container head structure is provided with corresponding internal threads, ensuring a secure and reliable connection.

Various modifications to these embodiments are apparent to those skilled in the art from the description and the accompanying drawings. The principles associated with the various embodiments described herein may be applied to other embodiments. Therefore, the description is not intended to be limited to the embodiments shown along with the accompanying drawings but is to provide the broadest scope consistent with the principles and the novel and inventive features disclosed or suggested herein. Accordingly, the invention is anticipated to hold on to all other such alternatives, modifications, and variations that fall within the scope of the present invention and appended claims.

1. A container head for dispensing liquid or semi-liquid compositions, comprising:
a head body having a liquid inlet end configured to be coupled to a liquid storage container and a liquid outlet end;
a liquid channel extending between the liquid inlet end and the liquid outlet end;
an elastic sealing structure mounted at or adjacent to the liquid outlet end, the elastic sealing structure is provided with a slit, the slit being normally closed to prevent leakage of liquid from the liquid storage container and elastically deformable to open under liquid pressure to permit flow through the liquid outlet end; and
a rolling ball applicator rotatably received in a groove at the liquid outlet end, the rolling ball being positioned to receive liquid discharged through the slit and to apply the liquid to a user's skin during rolling contact.

2. The container head of claim 1, wherein the slit is selected from a linear cut, a cross cut, a T-shaped cut, or a V-shaped cut.

3. The container head of claim 1, wherein the elastic sealing structure comprises a flexible elastomeric material.

4. The container head of claim 1, wherein the elastic sealing structure comprises a blocking portion integrally formed with a mounting portion, the blocking portion enclosing the slit that opens under liquid pressure and closes when the pressure is removed.

5. The container head of claim 1, wherein the elastic sealing structure comprises a sleeve having an end wall with the slit that elastically deforms under pressure to permit liquid flow and returns to a closed state when the pressure is removed.

6. The container head of claim 5, wherein the slit is provided on the sleeve of the elastic sealing structure.

7. The container head of claim 1, wherein the liquid channel further comprises a second elastic sealing structure positioned upstream of the elastic sealing structure to prevent backflow toward the liquid storage container.

8. The container head of claim 1, wherein the ball applicator is mounted in a spherical groove formed in a ball seat of the main body and configured to roll relative to the spherical groove.

9. A multifunctional applicator system comprising:
a container head having a liquid inlet end, a liquid outlet end, and a liquid channel extending therebetween;
an elastic sealing structure disposed at or adjacent to the liquid outlet end, the elastic sealing structure being normally closed to prevent liquid leakage and elastically deformable to permit liquid flow under applied pressure;
a rolling ball applicator rotatably mounted at the liquid outlet end in fluid communication with the elastic sealing structure for dispensing liquid onto a user's skin; and
a detachable cover configured to press the rolling ball against the elastic sealing structure when fitted, thereby reinforcing sealing during storage and transport.

10. The multifunctional applicator system of claim 9, wherein the cover includes an inner protrusion configured to press the rolling ball into the elastic sealing structure.

11. The multifunctional applicator system of claim 9, wherein the container head further comprises an outer shell and an inner shell assembled to define a cavity for accommodating internal components.

12. The multifunctional applicator system of claim 9, further comprising at least one stimulation element selected from a phototherapy lamp and a vibration motor.

13. The multifunctional applicator system of claim 9, wherein the elastic sealing structure comprises an elastic flap oriented toward the ball applicator to promote rapid closure after liquid pressure is released.

14. The multifunctional applicator system of claim 9, wherein the elastic sealing structure comprises a flexible sleeve having an end wall with a slit selected from a straight line slit, cross-shaped slit, or T-shaped slit.

15. The multifunctional applicator system of claim 14, wherein the slit is provided on the sleeve of the elastic sealing structure.

16. The multifunctional applicator system of claim 9, further comprising a second elastic sealing structure disposed upstream of the liquid outlet to provide dual leakage prevention.

17. A method of dispensing and applying a liquid skincare composition using a container head, the method comprising:
coupling the container head to a liquid storage container at a liquid inlet end;
applying pressure to the liquid storage container such that liquid flows into a liquid channel of the container head;
elastically deforming an end wall of an elastic sealing structure under liquid pressure to open a slit and discharge the liquid toward a rolling ball applicator; and
rolling the rolling ball applicator against a user's skin to transfer and spread the liquid thereon.

18. The method of claim 17, wherein applying pressure to the liquid storage container comprises squeezing a flexible bottle.

19. The method of claim 17, wherein the rolling ball applicator simultaneously massages the skin while transferring the liquid.

20. The method of claim 17, further comprising covering the rolling ball applicator with a detachable cover after use to mechanically reseal the slit of the elastic sealing structure.
